(19) United States
(12) Patent Application Publication
(10) Pub. No.: US 2016/0225145 A1
(43) Pub. Date: Aug. 4, 2016

(54) MEDICAL IMAGE PROCESSING DEVICE, MEDICAL IMAGE PROCESSING METHOD AND COMPUTER READABLE MEDIUM

(71) Applicant: ZIOSOFT, INC., Tokyo (JP)

(72) Inventor: Tsuyoshi NAGATA, Tokyo (JP)

(21) Appl. No.: 14/997,807

(22) Filed: Jan. 18, 2016

(30) Foreign Application Priority Data
Jan. 30, 2015 (JP) 2015-017810

(51) Int. Cl. G06T 7/00; A61B 6/00; G06K 9/32; A61B 6/03; G06K 9/46; G06K 9/62

(52) U.S. Cl. CPC G06T 7/0012; G06K 9/46; G06K 9/6215; G06K 9/3216; A61B 6/03; A61B 6/5217; G06T 2207/30028; G06T 2207/10021; G06T 2207/10028; G06T 2207/10081; G06T 2207/10068; G06K 2009/3225

(57) ABSTRACT

A medical image processing device includes an acquisition unit acquiring first and second volume data, an image deriving unit deriving first and second images based on the first and second volume data, a setting unit, an input unit and a report generating unit. The setting unit sets first, second and third marks for first, second and third feature parts included in the first and second volume data, and sets a correspondence relationship of the first and second marks corresponding to each other. The input unit inputs first finding information about the first and second feature parts, and inputs second finding information about the third feature part. The report generating unit generates a finding report which includes the first and second images, the first, second and third marks, the first and second finding information, and in which the first and third marks are displayed in a different expression.

### CROSS-REFERENCE TO RELATED APPLICATION

This application claims priority based on Japanese Patent Application No. 2015-017810, filed on Jan. 30, 2015, the entire contents of which are incorporated by reference herein.

### BACKGROUND OF THE INVENTION

1. Field of the Invention

The present invention relates to a medical image processing device, a medical image processing method, and a computer readable medium.

2. Related Art

In the related art, a radiologist or a radiological technologist makes an image diagnosis using various medical images according to requests from doctors in charge of patients and advises the doctors of policies of various inspections or therapy. Examples of the medical images include computed tomography (CT) images, magnetic resonance imaging (MRI) images, and ultrasonographic images. The radiologist or the radiological technologist generates documents (also referred to as a finding report or an image interpretation report) including findings from the image diagnosis and sends the generated document to the doctors in charge.

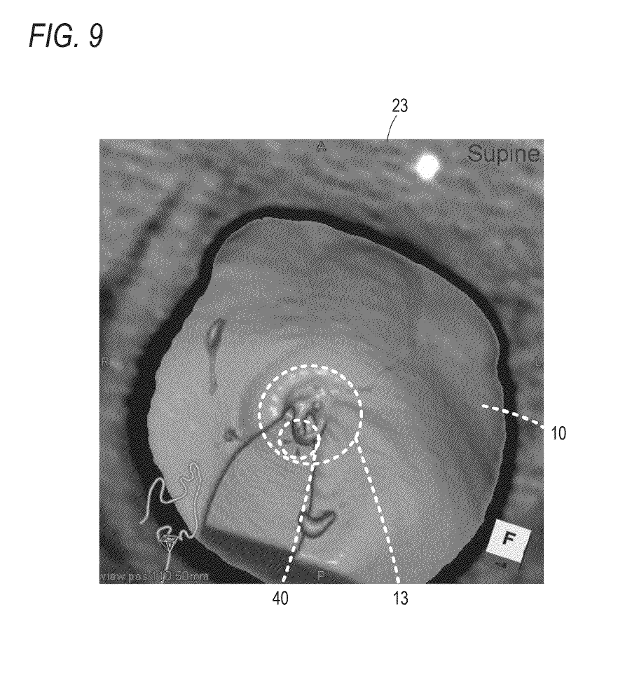

An image interpretation report display device is known which displays a sheet of inspection image (MRI image) of an inspected region and original sentences of a document based on the inspection result (for example, see US 2007/0237375A (FIG. 9)).

### SUMMARY OF THE INVENTION

In the image interpretation report display device described in US2007/0237375A, there is a possibility that accuracy or quality of image interpretation (image interpretation accuracy) from which can be interpreted from inspection image data will not be satisfactory.

The present invention is made for the above-mentioned circumstances and provides a medical image processing device, a medical image processing method, and a computer readable medium storing a medical image processing program which can improve image interpretation accuracy using a medical image.

A medical image processing device of the present invention includes an acquisition unit, an image deriving unit, a setting unit, an input unit and a report generating unit. The acquisition unit acquires first volume data including a digestive organ which is imaged in a first body position and acquires second volume data including the digestive organ which is imaged in a second body position. The image deriving unit derives a first image including the digestive organ based on the first volume data and derives a second image including the digestive organ based on the second volume data. The setting unit sets a first mark for a first feature part included in the first volume data, sets a second mark for a second feature part included in the second volume data, sets a third mark for a third feature part included in the second volume data, and sets a correspondence relationship indicating that the first mark and the second mark correspond to each other. The input unit inputs first finding information based on a common finding about the first feature part having the first mark and the second feature part having the second mark. The first and second marks are set to the correspondence relationship. The input unit inputs second finding information based on an individual finding about the third feature part having the third mark. The report generating unit generates a finding report which includes the first image, the second image, the first and second marks set to the correspondence relationship, the first finding information, the third mark, and the second finding information, in which the first mark and the second mark are displayed in a same expression, and in which the first mark and the third mark are displayed in a different expression.

A medical image processing method of the present invention in a medical image processing device, includes: acquiring first volume data including a digestive organ which is imaged in a first body position; acquiring second volume data including the digestive organ which is imaged in a second body position; deriving a first image including the digestive organ based on the first volume data; deriving a second image including the digestive organ based on the second volume data; setting a first mark for a first feature part included in the first volume data; setting a second mark for a second feature part included in the second volume data; setting a third mark for a third feature part included in the second volume data; setting a correspondence relationship indicating that the first mark and the second mark correspond to each other; inputting first finding information based on a common finding about the first feature part having the first mark and the second feature part having the second mark, the first and second marks being set to the correspondence relationship; inputting second finding information based on an individual finding about the third feature part having the third mark; and generating a finding report which includes the first image, the second image, the first and second marks set to the correspondence relationship, the first finding information, the third mark, and the second finding information, in which the first mark and the second mark are displayed in a same expression, and in which the first mark and the third mark are displayed in a different expression.

A non-transitory computer readable medium stores program for causing a medical image processing device to execute operations including: acquiring first volume data including a digestive organ which is imaged in a first body position; acquiring second volume data including the digestive organ which is imaged in a second body position; deriving a first image including the digestive organ based on the first volume data; deriving a second image including the digestive organ based on the second volume data; setting a first mark for a first feature part included in the first volume data; setting a second mark for a second feature part included in the second volume data; setting a third mark for a third feature part included in the second volume data; setting a correspondence relationship indicating that the first mark and the second mark correspond to each other; inputting first finding information based on a common finding about the first feature part having the first mark and the second feature part having the second mark, the first and second marks being set to the correspondence relationship; inputting second finding information based on an individual finding about the third feature part having the third mark; and generating a finding report which includes the first image, the second image, the first and second marks set to the correspondence relationship, the first finding information, the third mark, and the second finding information, in which the first mark and the second mark are displayed in a same expression, and in which the first mark and the third mark are displayed in a different expression.

According to the present invention, it is possible to improve image interpretation accuracy using a medical image.

### BRIEF DESCRIPTION OF THE INVENTION

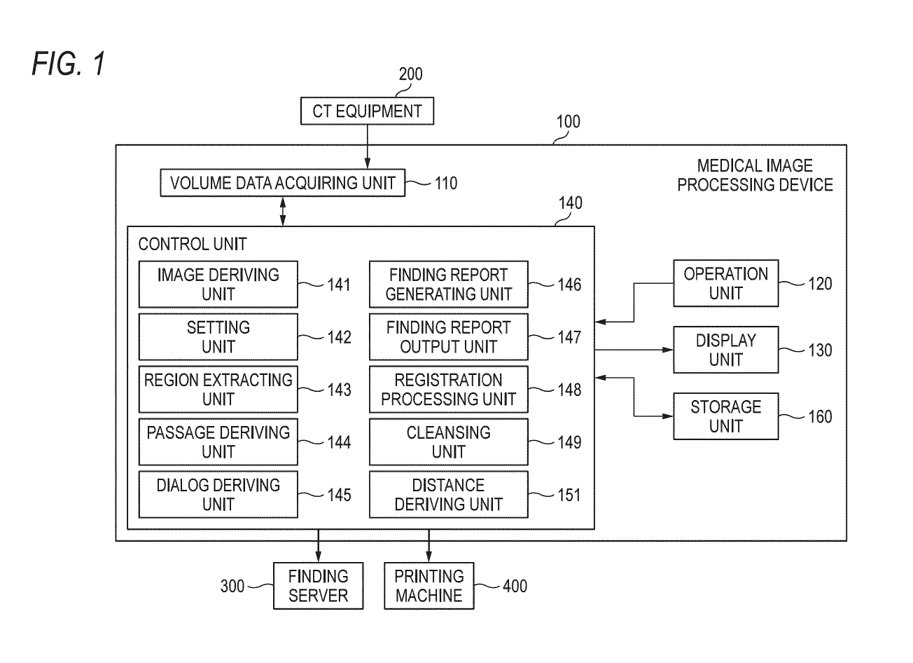

FIG. 1 is a block diagram illustrating a configuration example of a medical image processing device according to an embodiment.

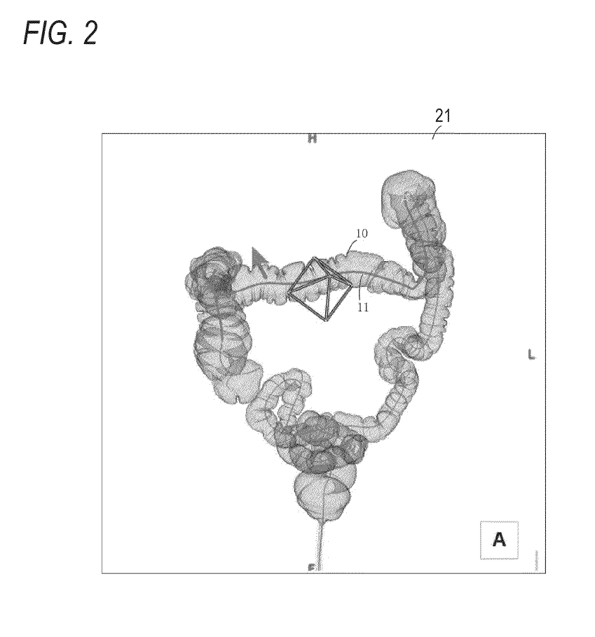

FIG. 2 is a schematic diagram illustrating an example of an overview image of a large intestine according to the embodiment.

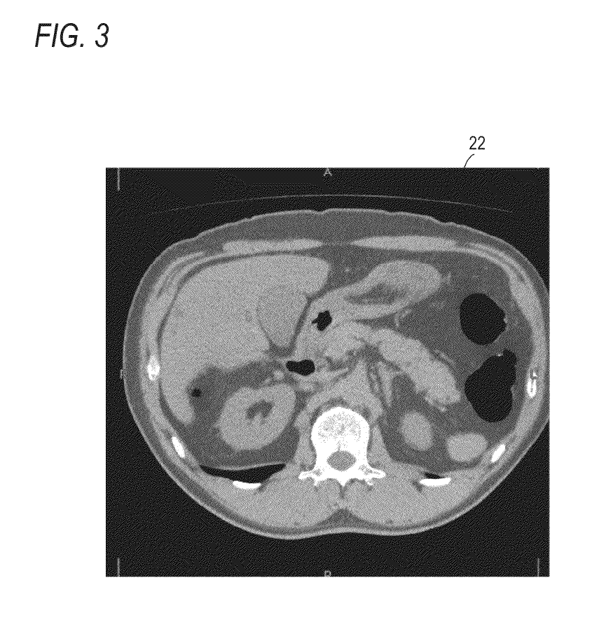

FIG. 3 is a schematic diagram illustrating an example of a multi-planar reconstruction (MPR) image of an abdomen according to the embodiment.

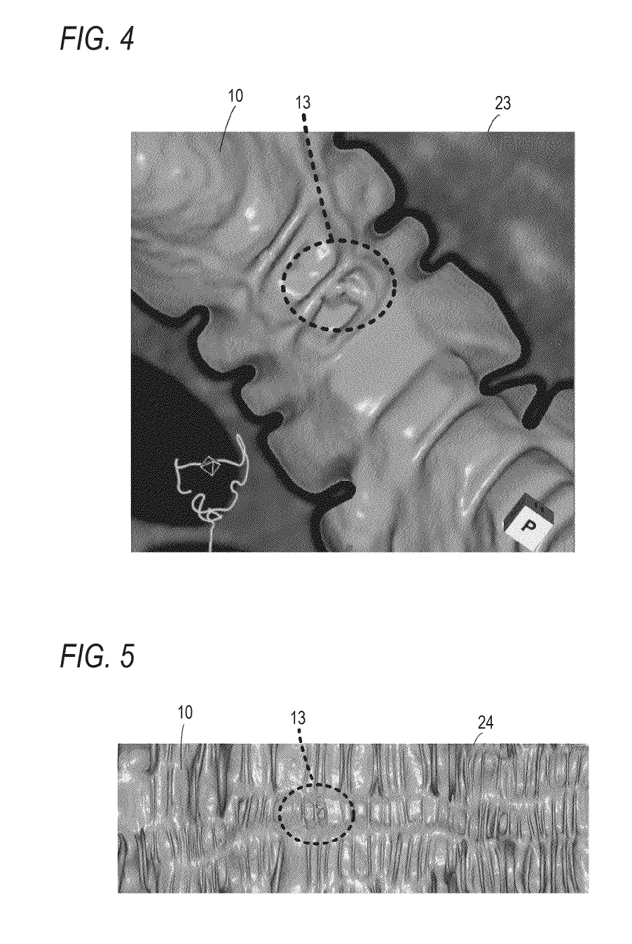

FIG. 4 is a schematic diagram illustrating an example of a virtual endoscopic image of a large intestine according to the embodiment.

FIG. 5 is a schematic diagram illustrating an example of a cylindrical projection image of a large intestine according to the embodiment.

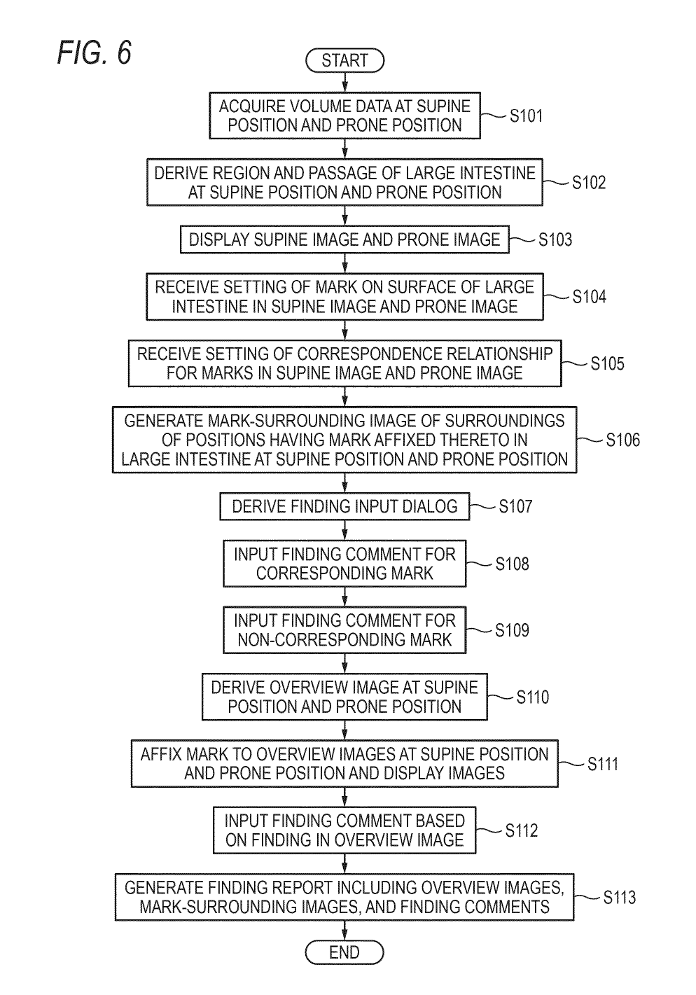

FIG. 6 is a flowchart illustrating an operation example of the medical image processing device according to the embodiment.

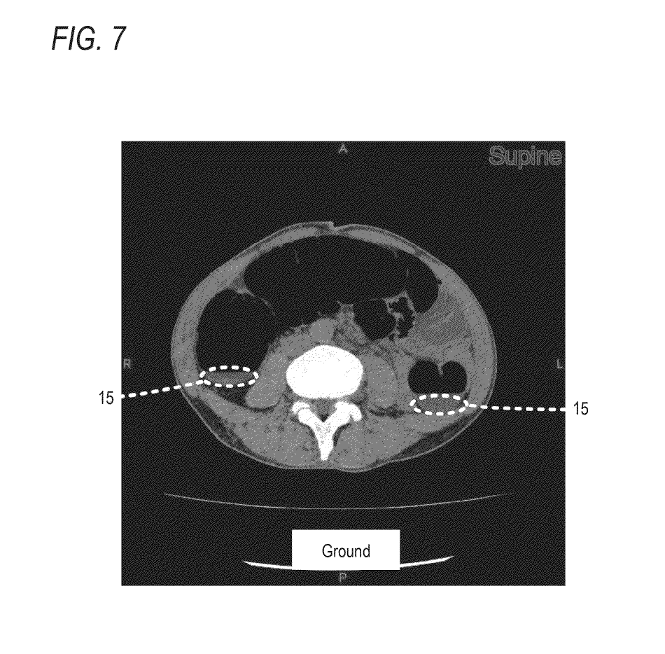

FIG. 7 is a schematic diagram illustrating an example of a sectional image of an abdomen in a CT image which is captured in a supine position according to the embodiment.

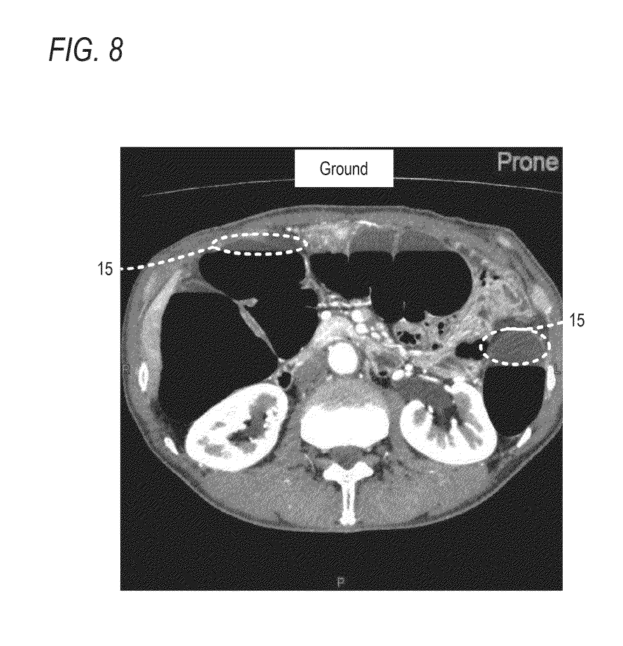

FIG. 8 is a schematic diagram illustrating an example of a sectional image of an abdomen in a CT image which is captured in a prone position according to the embodiment.

FIG. 9 is an enlarged view illustrating an example of a virtual endoscopic image having a mark affixed thereto according to the embodiment.

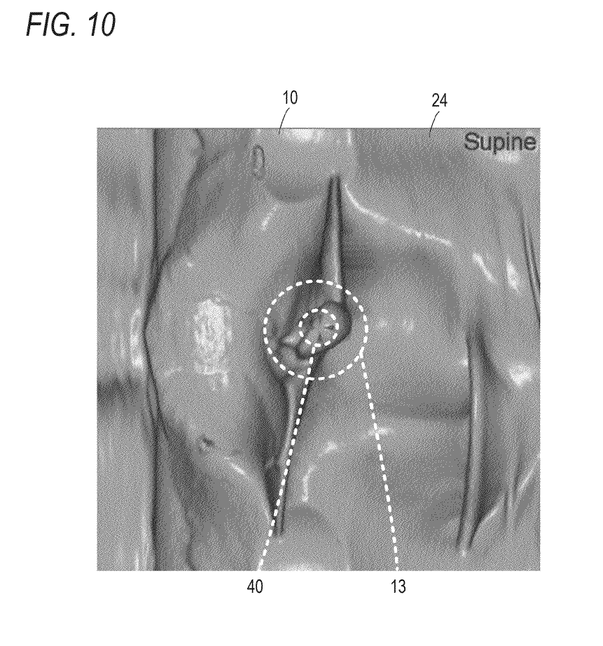

FIG. 10 is an enlarged view illustrating an example of a cylindrical projection image having a mark affixed thereto according to the embodiment.

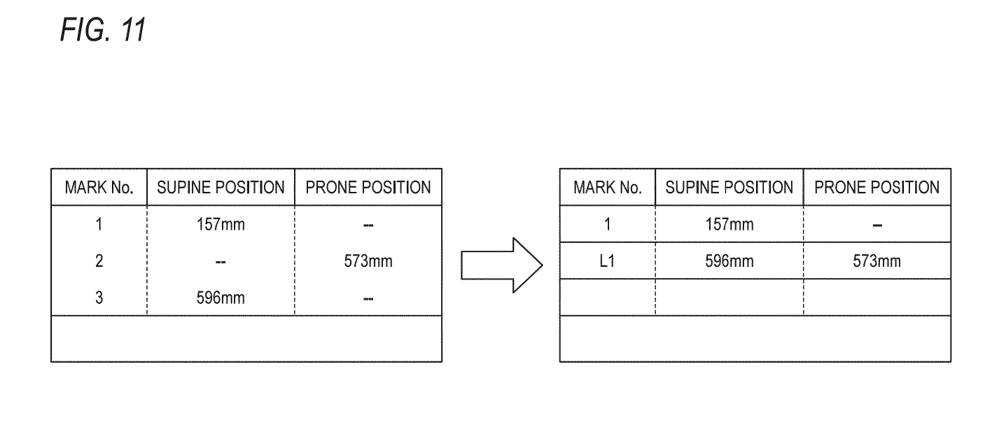

FIG. 11 is a schematic diagram illustrating an example of distances from an anus to marks at a supine position and a prone position according to the embodiment.

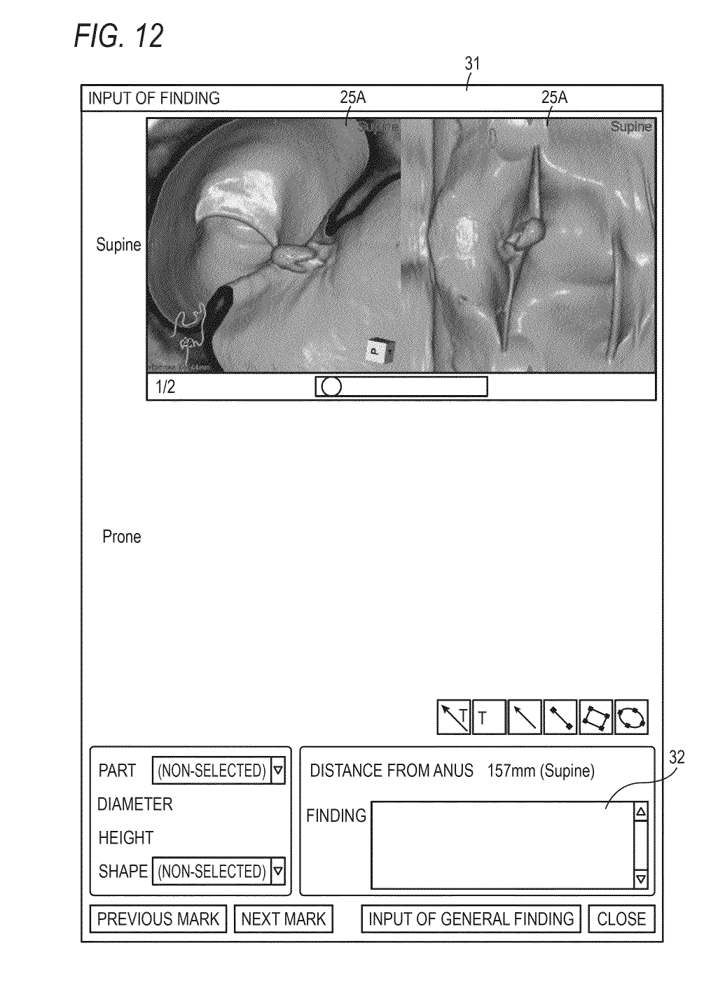

FIG. 12 is a schematic diagram illustrating an example of a finding input dialog including a mark-surrounding image when marks are affixed to a supine image and marks are affixed to a prone image.

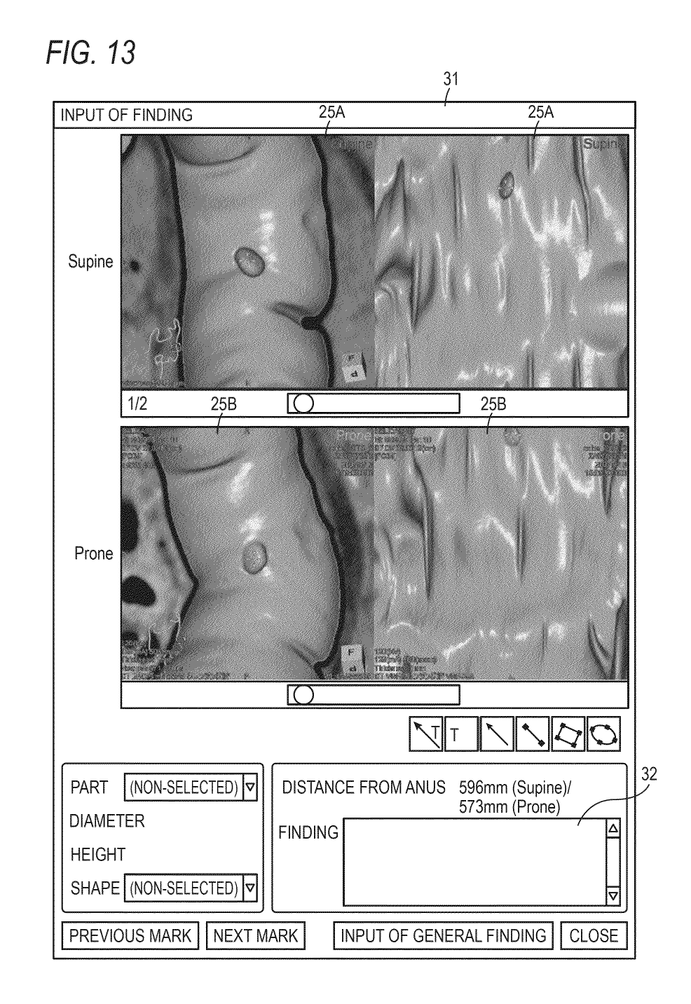

FIG. 13 is a schematic diagram illustrating an example of a finding input dialog including mark-surrounding images when marks are affixed to a supine image and a prone image.

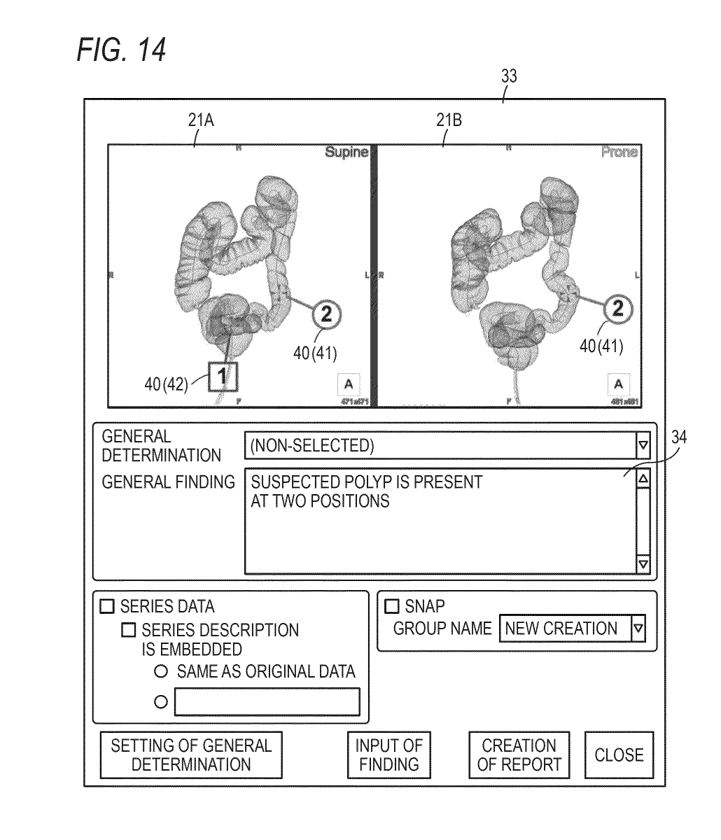

FIG. 14 is a schematic diagram illustrating an example of a finding input dialog including an overview image according to the embodiment.

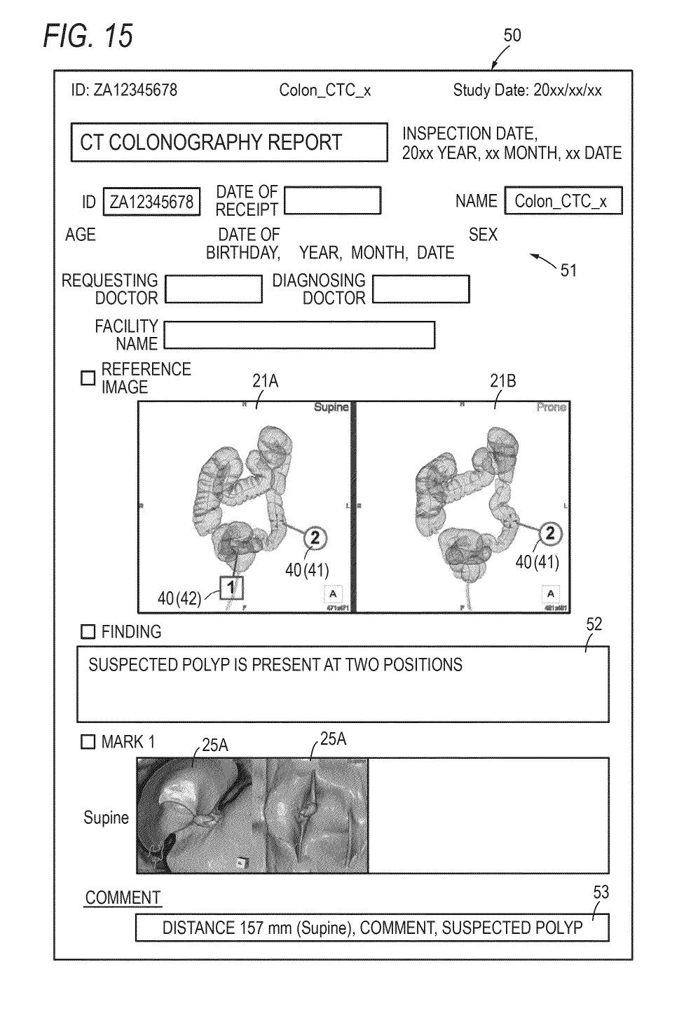

FIG. 15 is a schematic diagram illustrating an example of a finding report according to the embodiment.

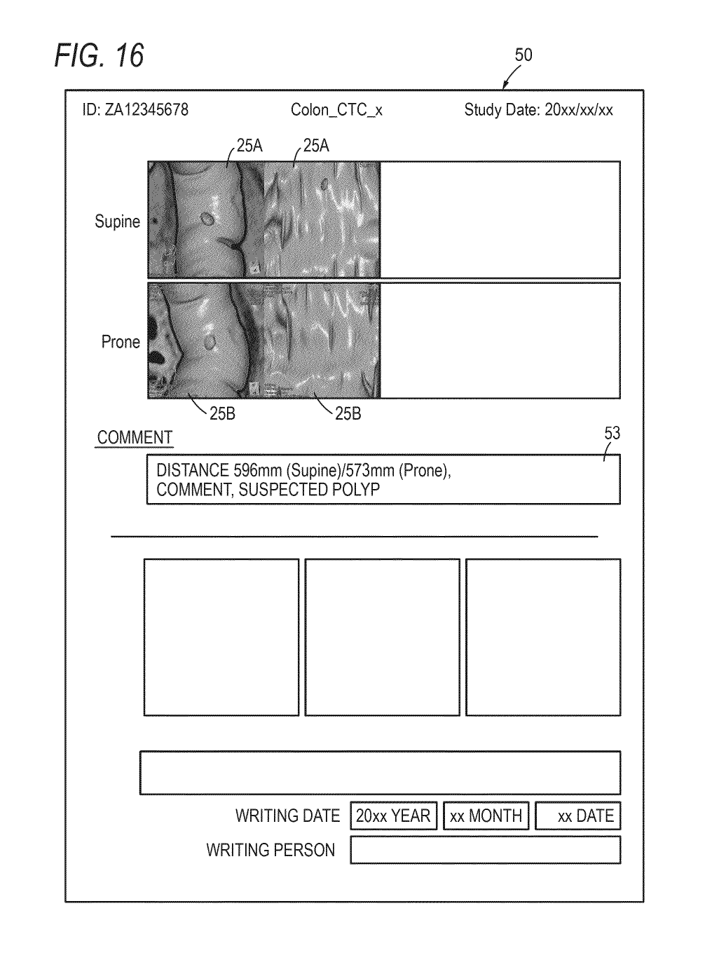

FIG. 16 is a schematic diagram (subsequent to FIG. 15) illustrating an example of the finding report according to the embodiment.

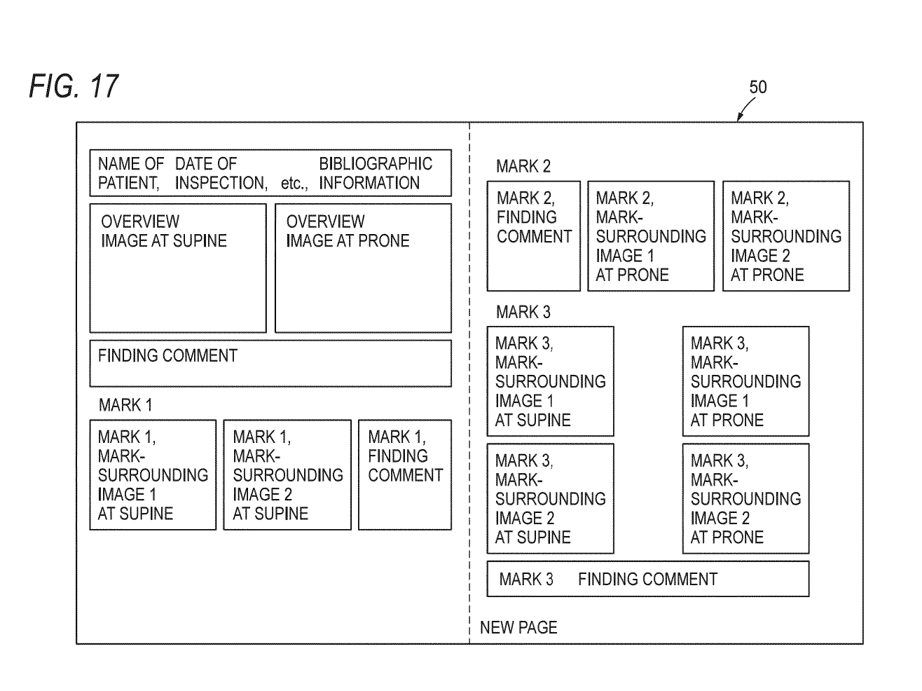

FIG. 17 is a schematic diagram illustrating another layout example of the finding report according to the embodiment.

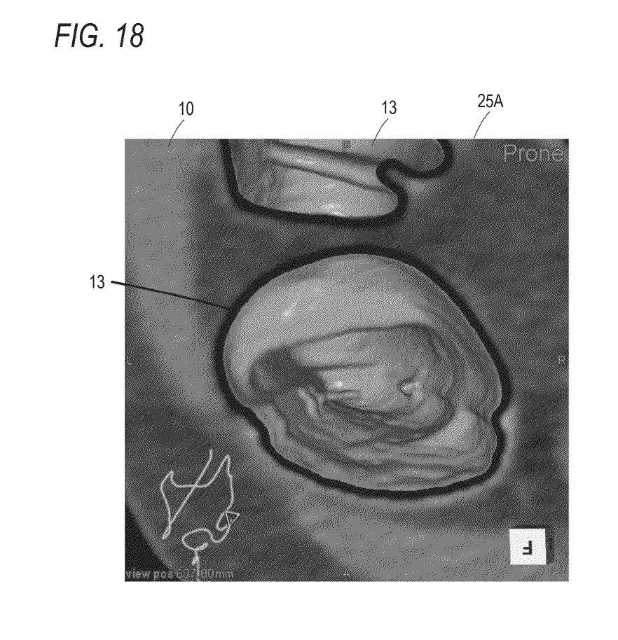

FIG. 18 is an enlarged view illustrating an example of a virtual endoscopic image subjected to a cleansing process according to the embodiment.

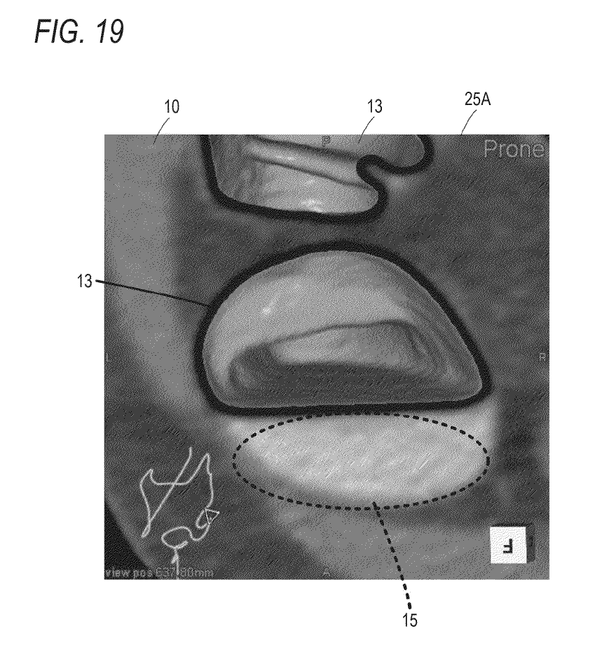

FIG. 19 is an enlarged view illustrating an example of a virtual endoscopic image not subjected to a cleansing process according to the embodiment.

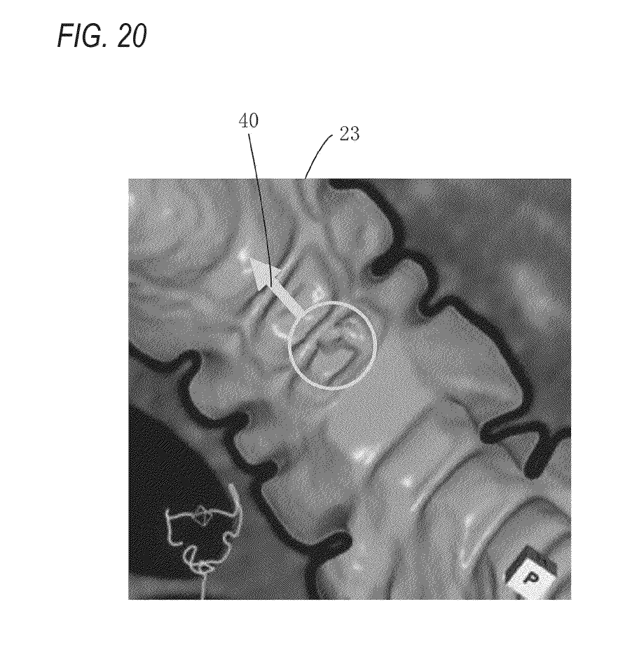

FIG. 20 is a schematic diagram illustrating an example of a mark image of a mark indicating an upstream side of a large intestine according to the embodiment.

### DETAILED DESCRIPTION OF THE INVENTION

Hereinafter, embodiments of the present invention will be described with reference to the accompanying drawings.

### Circumstances Leading to Embodiment

In CT colonography, a virtual endoscopic image can be obtained by processing a captured image. The CT colonography does not use an endoscope and is thus less invasive. For example, when the image interpretation report display device described in US2007/0237375A is applied to display of a result of a large intestine inspection, a virtual endoscopic image of the large intestine and a comment of a document foment based on the inspection result of the large intestine are added to the display.

In the CT colonography, in general, dietary restriction or the like is carried out before inspection to easily visually recognize tissues in the large intestine, but residues may be present in the large intestine. When a residue is present in the large intestine, the shape of the large intestine viewed in one direction can be grasped from a virtual endoscopic image of the large intestine, but it is difficult to grasp the shape underneath the residue. Accordingly, in the CT colonography, the large intestine is imaged at two body positions of a supine position and a prone position and the residue is made to flow, thereby thoroughly performing an inspection. On the other hand, a discrepancy may occur between two imaging results, thereby lowering image interpretation accuracy.

Hereinafter, a medical image processing device, a medical image processing method, and a medical image processing program which can improve image interpretation accuracy using a medical image.

In the present invention, a medical image processing device includes at least one processor, at least one memory and a display unit. At least one processor functions as an acquisition unit, an image deriving unit, a setting unit, an input unit and a report generating unit. The acquisition unit acquires first volume data including a digestive organ which is imaged in a first body position and acquires second volume data including the digestive organ which is imaged in a second body position. The image deriving unit derives a first image including the digestive organ based on the first volume data and derives a second image including the digestive organ based on the second volume data. The setting unit sets a first mark for a first feature part included in the first volume data, sets a second mark for a second feature part included in the second volume data, sets a third mark for a third feature part included in the second volume data, and sets a correspondence relationship indicating that the first mark and the second mark correspond to each other. The input unit inputs first finding information based on a common finding about the first feature part having the first mark and the second feature part having the second mark. The first and second marks are set to the correspondence relationship. The input unit inputs second finding information based on an individual finding about the third feature part having the third mark. The report generating unit generates a finding report which includes the first image, the second image, the first and second marks set to the correspondence relationship, the first finding information, the third mark, and the second finding information, in which the first mark and the second mark are displayed in a same expression, and in which the first mark and the third mark are displayed in a different expression. At least one memory may store the first and second volume data, the first and second images, the first, second and third marks, the first, second and third feature parts, the correspondence relationship, the first and second finding information and the finding report. The display unit may display at least the finding report.

### Embodiment

FIG. 1 is a block diagram illustrating a configuration example of a medical image processing device 100 according to an embodiment. The medical image processing device 100 includes a volume data acquiring unit 110, an operation unit 120, a display unit 130, a control unit 140, and a storage unit 160. The medical image processing device 100 is connected to CT equipment 200 a finding server 300, and a printing machine 400. The medical image processing device 100 acquires volume data from the CT equipment 200 and processes the acquired volume data.

The control unit 140 includes an image deriving unit 141, a setting unit 142, a region extracting unit 143, a passage deriving unit 144, a dialog deriving unit 145, a finding report generating unit 146, a finding report output unit 147, a registration processing unit 148, a cleansing unit 149, and a distance deriving unit 151. The registration processing unit 148 and the cleansing unit 149 may be omitted.

The control unit 140 includes a central processing unit (CPU) or a digital signal processor (DSP). The control unit 140 includes a read only memory (ROM) or a random access memory (RAM). The CPU or the DSP realizes the functions of the control unit 140 by executing a medical image processing program stored in the ROM or the RAM.

The CT equipment 200 irradiates a living body with X-rays and captures an image (CT image) using a difference in absorption of X-rays between tissues in the living body. Plural CT images may be captured in a time-series manner. The CT image forms volume data including information of an arbitrary place in the living body. By capturing the CT image, pixel values (CT values) of pixels (voxels) in the CT image are obtained. The CT equipment 200 transmits the volume data as the CT image to the medical image processing device 100 via a wired line or a wireless line. In this embodiment, the CT image includes at least an image of a large intestine.

Since the volume data as the captured CT image is stored for the time being, the CT image may be transmitted to and stored in a server or the like on a network. In this case, the volume data acquiring unit 110 of the medical image processing device 100 may acquire the volume data as needed.

The volume data acquiring unit 110 acquires the volume data as the CT image. The volume data acquiring unit 110 may acquire the volume data from the CT equipment 200 or the server on the network by communication via a wired line or a wireless line or may acquire the volume data via an arbitrary storage medium (not illustrated). The volume data acquiring unit 110 is an example of the acquisition unit.

The acquired volume data may be immediately sent to and variously processed by the control unit 140, or may be stored in the storage unit 160 and then may be sent to and various processed by the control unit 140 as needed.

The operation unit 120 includes, for example, a touch panel, a pointing device, and a keyboard. The operation unit 120 receives an arbitrary input operation from a user (for example, a doctor, a radiological technologist, or a radiologist) of the medical image processing device 100. The operation unit 120 is an example of the input unit that inputs finding information.

The display unit 130 includes, for example, a liquid crystal display (LCD) and displays a variety of information. The display unit 130 displays various images (such as an overview, a multi-planar reconstruction (MPR) image, a virtual endoscopic image, and a cylindrical projection image). In addition, the display unit 130 displays various windows (for example, a mark affixing window for affixing a mark, a correspondence relationship setting window for setting a correspondence relationship between marks, a finding input dialog for inputting a finding comment, and a report display dialog for displaying a finding report). The display unit 130 is an example of the output unit that outputs a variety of warning information.

The storage unit 160 stores information (for example, various images derived by the image deriving unit 141, setting information set by the setting unit 142, and information of various screens derived by the screen deriving unit 145) derived by the control unit 140. The storage unit 160 also stores volume data, a variety of data, various programs, and a variety of other information.

The image deriving unit 141 derives various images from the volume data acquired by the volume data acquiring unit 110 based on various rendering method. The image deriving unit 141 may derive various images using the volume data of a large intestine 10 after a region of the large intestine 10 is extracted by the region extracting unit 143. The large intestine 10 in the drawings includes a colon, but since both are practically diagnosed together, the large intestine and the colon are not particularly distinguished from each other in this embodiment.

The image deriving unit 141 may generate a three-dimensional image from the volume data using volume rendering based on a known method. FIG. 2 is a schematic diagram illustrating an example of an overview image 21 of the large intestine 10 as a three-dimensional image.

The image deriving unit 141 derives an MPR image 22 as a two-dimensional image from the volume data based on a known method (for example, a method described in US 2006/0056681 A). FIG. 3 is a schematic diagram illustrating an example of an MPR image 22 of an abdomen. The MPR image 22 is a sectional image (two-dimensional image) indicating a state cut by an arbitrary sectional plane. Examples of the arbitrary sectional plane include a body-axis sectional plane, an arrow-like sectional plane, a coronal sectional plane, and an oblique sectional plane.

The image deriving unit 141 derives a virtual endoscopic image 23 as a two-dimensional image from the volume data using a perspective projection method based a known method (for example, see US 2008/0075346 A). FIG. 4 is a schematic diagram illustrating an example of a virtual endoscopic image 23 of the large intestine 10. The virtual endoscopic image can be acquired using a parallel projection method or a perspective projection method.

The image deriving unit 141 derives a cylindrical projection image 14 as a two-dimensional image from the volume data based a known method (for example, see US 2007/0120845). FIG. 5 is a schematic diagram illustrating an example of a cylindrical projection image 24 of the large intestine 10. The cylindrical projection image 24 illustrated in FIG. 5 corresponds to an image obtained by exfoliating the large intestine 10 with respect to a reference line (a central line 11 of the large intestine 10 which is marked in the overview image 21 of FIG. 2). The reference line of the large intestine 10 is not limited to the central line 11, but may be a line indicating a passage of the large intestine.

When an input operation is received by the operation unit 120, the setting unit 142 performs various settings based on the input information. The setting information is stored in the storage unit 160.

The region extracting unit 143 extracts an arbitrary region from the volume data in the living body including the arbitrary region based on a known method. In this embodiment, for example, the large intestine 10 is extracted, but another digestive organ (for example, a small intestine, a stomach, and a gullet) may be extracted. This is because a residue may be included in these organs at the time of imaging. The region extracting unit 143 extracts as the region of the large intestine 10 including the region of the residue 15. The region extracted by the region extracting unit 143 is used, for example, to improve display quality of various images or to generate the central line 11 of the large intestine 10. The region extracting unit 143 is an example of the extraction unit that extracts the residue 15.

The passage deriving unit 144 derives the passage of the large intestine 10 from the entire volume data or from the volume data based on the large intestine 10 using a known method.

The passage deriving unit 144 derives the central line 11 of the large intestine 10. The passage deriving unit 144 can acquire the central line 11, for example, by extracting a region of air and the residue 15 in the large intestine and performing a thinning process thereon. A post-process such as smoothing may be further performed thereon. The central line 11 can be used as the passage of the large intestine 10. The passage deriving unit 144 is an example of the reference line deriving unit.

The screen deriving unit 145 derives various screens based on screen layout information, information input by the operation unit 120, and the images derived by the image deriving unit 141. The derived screens include a finding input screen (for example, see FIGS. 12 to 14) and a screen on which a finding report 50 is displayed (see FIGS. 15 to 17)).

The screen layout information is stored in the storage unit 160. When plural screen layouts are present, an input operation for selecting a screen layout may be received by the operation unit 120 and the screen layout may be set by the setting unit 142.

The finding report generating unit 146 generates a finding report based on layout information of the finding report and information input to the finding input screen. The finding report is confirmed by a user of the medical image processing device 100 or the like. The finding report generating unit 146 is an example of the report generating unit.

The finding report output unit 147 outputs the derived finding report to the display unit 130, the finding server 300, or the printing machine 400. The finding report output unit 147 outputs the finding report in the layout which has been determined in the stage of the finding report generating unit 146. Accordingly, the visibility of the finding report is maintained.

The registration processing unit 148 performs registration on plural images (two-dimensional images or three-dimensional images). In the registration, relevant regions are registrated. In this case, the registration processing unit 148 may perform three-dimensional registration in consideration of three-dimensional images or may perform two-dimensional registration in consideration of two-dimensional images.

The registration processing unit 148 performs registration on plural images. Details of the registration will be described later.

The cleansing unit 149 virtually removes the region of the residue 15 in the large intestine 10 extracted by the region extracting unit 143. Details of the cleansing process (virtual cleaning) will be described later.

The distance deriving unit 151 derives a distance between plural predetermined positions in the large intestine 10 and a distance between a predetermined point and a predetermined position in the large intestine 10 based on the volume data or the passage of the large intestine 10.

The medical image processing device 100 is connected to the finding server 300 and the printing machine 400 via a network and the like.

The finding server 300 acquires and stores the finding report generated by the medical image processing device 100. The finding server 300 is connected to a wireless network or a wired network and is configured to be accessed by the devices. The finding reports stored in the finding server 300 are acquired via the network by a personal computer (PC) (not illustrated) which is used by a doctor carrying out an examination in an examination room, are displayed on the screen thereof, and are used as diagnosis materials of the doctor.

The finding server 300 may store electronic medical records or other information along with the finding reports. At least some information in the finding reports may be used as the electronic medical records or the other information.

The printing machine 400 prints the finding report generated by the medical image processing device 100 on a medium such as a paper sheet.

An operation example of the medical image processing device 100 will be described below.

FIG. 6 is a flowchart illustrating an operation example of the medical image processing device 100.

The CT equipment 200 captures a CT image including the vicinity of the large intestine 10 of a human body such as a patient and acquires volume data. Here, the CT equipment 200 captures a CT image at a supine position and a prone position and acquires volume data at the supine position and volume data at the prone position.

When the CT images are captured, for example, a patient or the like is subjected to capturing a CT image at the supine position, and then the body thereof is rotated to the prone position in the CT equipment 200 and is subjected to capturing a CT image at the prone position. Accordingly, a slight time difference is present between the imaging time of the CT image at the supine position and the imaging time of the CT image at the prone position.

A CT image may be captured at another position (for example, a lateral recumbent position) by the CT equipment 200 to acquire volume data.

FIG. 7 is a schematic diagram illustrating an example of a sectional image (MPR image) as a CT image at the supine position. FIG. 8 is a schematic diagram illustrating an example of a sectional image (MPR image) as a CT image at the prone position. In FIGS. 7 and 8, the “Ground” side represents the downside in the vertical direction. FIGS. 7 and 8 illustrate an appearance difference at the same position of a human body between the supine position and the prone position and do not intend the CT equipment to actually generate the MPR images in S101.

In FIGS. 7 and 8, the positions surrounded by broken lines denote residues and the residues appear in both drawings. In FIGS. 7 and 8, it can be understood that the position of the sectional plane of the CT image is almost the same in a human body, but the position and shape of a residue in the CT image slightly differ.

In the medical image processing device 100, the volume data acquiring unit 110 acquires volume data at the supine position and volume data at the prone position from the CT equipment 200 (S101).

The region extracting unit 143 extracts the region of the large intestine 10 at the supine position from the volume data at the supine position (S102). The region extracting unit 143 extracts the region of the large intestine 10 at the prone position from the volume data at the prone position (S102). The region of the large intestine 10 can include a region of the residue 15.

The passage deriving unit 144 derives the passage of the large intestine 10 at the supine position from the volume data at the supine position (S102). The passage deriving unit 144 derives the passage of the large intestine 10 at the prone position from the volume data at the prone position (S102).

The image deriving unit 141 derives a supine image of the large intestine 10 from the volume data of the large intestine 10 at the supine position based on the region and the passage of the large intestine 10 at the supine position. The supine image is an image based on the supine position and examples thereof include a virtual endoscopic image 23 of the large intestine 10 at the supine position, a cylindrical projection image 24 of the large intestine 10 at the supine position, and other images from which the shape of the large intestine at the supine position can be observed.

The image deriving unit 141 derives a prone image of the large intestine 10 from the volume data of the large intestine 10 at the prone position based on the region and the passage of the large intestine 10 at the prone position. The prone image is an image based on the prone position and examples thereof include a virtual endoscopic image 23 of the large intestine 10 at the prone position, a cylindrical projection image 24 of the large intestine 10 at the prone position, and other images from which the shape of the large intestine at the prone position can be observed.

The display unit 130 displays the derived supine image and the derived prone image (S103). Here, the display unit 130 may display the supine image and the prone image individually or may display the supine image and the prone image as a set.

The operation unit 120 receives an input operation for affixing a mark to a feature part 13 in the large intestine 10 in the supine image displayed on the display unit 130 (S104). Similarly, the operation unit 120 receives an input operation for affixing a mark to a feature part 13 in the large intestine 10 in the prone image (S104).

The feature part 13 is a part which a user determines to have a feature in comparison with other parts in the image displayed on the display unit 130. The feature part 13 is a part determined to be a disease, a part suspected to be a disease, or a part attracting a user's interest. Examples of the disease include a tumor and a polyp.

The operation of affixing a mark 40 is input via a mark input window (not illustrated). On the mark input window, for example, an MPR image 22, a virtual endoscopic image 23, a cylindrical projection image 24, and an overview image 21 are displayed. When the operation of affixing the mark 40 is received by the operation unit 120, the setting unit 142 affixes the mark 40 to the feature part 13 in the supine image and affixes the mark 40 to the feature part 13 in the prone image. Information having the mark affixed thereto is stored as setting information in the storage unit 160. Here, the feature part 13 is marked by a user, but the feature part 13 may be automatically set in the image interpretation. In this case, it can be considered that the set feature part 13 is screened by the user. The feature part 13 may be expressed as a point or may be expressed as a surface or a region.

When the mark 40 is affixed, for example, the mark 40 is displayed on the display unit 130. FIG. 9 is a zoomed view illustrating an example of the virtual endoscopic image 23 having the mark 40 affixed thereto. FIG. 10 is a zoomed view illustrating an example of the cylindrical projection image 24 having the mark 40 affixed thereto.

Since the supine image and the prone image are images acquired by imaging the large intestine 10 at different positions from different points of view, there is a possibility that the same disease appears in both images. The user observes plural marks 40 in the images displayed on the display unit 130 and determines whether plural feature parts 13 having the marks 40 affixed thereto have a correspondence relationship. This correspondence relationship includes a relationship in which the positions or the shapes of the feature parts 13 are similar to each other and the plural feature parts represent the same disease and a relationship in which the plural feature pans 13 represent relevant diseases.

When the user determines that the marks 40 affixed to the feature parts 13 having a correspondence relationship are present in the marks 40 affixed to the feature parts 13 in the supine image and the marks 40 affixed to the feature parts 13 in the prone image, the user performs an operation of setting the correspondence relationship for the plural marks having the correspondence relationship.

The correspondence relationship setting operation is performed subsequently to the operation of affixing the mark 40 using the mark input window (not illustrated). The operation unit 120 receives the correspondence relationship setting operation from the user (S105). When the correspondence relationship setting operation is received, the setting unit 142 sets the correspondence relationship for the plural marks and stores the setting information in the storage unit 160. The setting information includes position information indicating the positions to which the marks 40 are affixed in the large intestine 10, that is, position information of the feature parts 13.

FIG. 11 is a schematic diagram illustrating an example of distances from an anus to the marks 40 in the volume data at the supine position and the prone position. These distances are derived by the distance deriving unit 151 based on the positions or the passages of the anus or the large intestine 10 included in the volume data.

In FIG. 11, two marks 40 (Mark No. 1 and No. 3) are affixed to the supine image, and one mark 40 (Mark No. 2) is affixed to the prone image. The distance information illustrated in FIG. 11 is displayed on the display unit 130.

When the user confirms the distance values on the display unit 130, the distance values (573 mm and 596 mm) of Mark No. 2 and Mark No. 3 are close to each other and thus are set as correspondence relationship setting candidates. The user confirms both images, recognizes both parts as the same target, performs the correspondence relationship setting operation, and the operation unit 120 receives this setting operation. In this case, the setting unit 142 may change the mark number to, for example, Mark No. L1.

The feature parts 13 having the marks 40 affixed thereto in the supine image and the prone image may not have any correspondence relationship with the feature parts 13 having other marks 40 affixed thereto. For example, in capturing the CT image at the supine position and capturing the CT image at the prone position, it is considered that the position or the shape of the large intestine 10 as a subject varies and does not appear in the other image or that underneath the residue 15 is not imaged due to the influence of the residue 15 and does not appear in the other image.

In this embodiment, the mark 40 subjected to the correspondence relationship setting operation is also referred to as “corresponding mark” and the mark 40 not subjected to the correspondence relationship setting operation is also referred to as “non-corresponding mark.” When the correspondence relationship setting operation is performed, the display unit 130 displays a corresponding mark 41 or a non-corresponding mark 42 as the mark 40.

In this way, in order to record the feature parts 13 of interest, the user performs an operation of affixing the mark 40 or an operation of setting the correspondence relationship. The user carefully observes the parts having the corresponding mark 41 or the non-corresponding mark 42 affixed thereto or the parts having the correspondence relationship set therefor in the supine image and the prone image. The user can determine or give an advice to transition to a next stage such as surgery as needed.

When the marks 40 are affixed in the supine image and the correspondence relationship is set, the image deriving unit 141 derives an enlarged image of the surrounding (mark-surrounding image 25) of the feature part 13 having the mark 40 affixed thereto in the large intestine 10 at the supine position based on the volume data at the supine position (S106). The mark-surrounding image 25 at the supine position is also referred to as a mark-surrounding image 25A. That is, the image deriving unit 141 has a function as a mark-surrounding image generating unit that generates a mark-surrounding image.

The mark-surrounding image 25A at the supine image is an image obtained by enlarging a part of the supine image and examples thereof include a virtual endoscopic image of the surrounding of the mark 40 at the supine position, a cylindrical projection image of the surrounding of the mark 40 at the supine position, and other images from which the shape of the surrounding of the mark 40 at the supine position can be observed.

When the marks 40 are affixed in the prone image and the correspondence relationship is set, the image deriving unit 141 derives an enlarged image of the surrounding (mark-surrounding image 25) of the feature part 13 having the mark 40 affixed thereto in the large intestine 10 at the prone position based on the volume data at the prone position (S106). The mark-surrounding image 25 at the prone position is also referred to as a mark-surrounding image 25B.

The mark-surrounding image 25B at the prone image is an image obtained by enlarging a part of the prone image and examples thereof include a virtual endoscopic image of the surrounding of the mark 40 at the prone position, a cylindrical projection image of the surrounding of the mark 40 at the prone position, and other images from which the shape of the surrounding of the mark 40 at the prone position can be observed.

It is preferable that the number of mark-surrounding images 25 generated be enough for the user to make a diagnosis. The mark-surrounding images 25 are attached to the finding report later. The mark-surrounding images 25 may include a diameter measurement result of a polyp therein.

The dialog deriving unit 145 derives a finding input dialog 31 based on the affixation information of the mark 40, the correspondence relationship setting information, and the mark-surrounding image 25 (S107). The finding input dialog 31 is a dialog which is used for the user to input a finding comment while observing the mark-surrounding image 25 or the like.

FIGS. 12 and 13 are schematic diagrams illustrating an example of the finding input dialog 31. The finding input dialog 31 is generated for each mark 40 and includes the mark-surrounding image 25A at the supine position, the mark-surrounding image 25B at the prone position, and a finding comment input box for the mark 40.

The finding comment for Mark No. 1 illustrated in FIG. 11 is input in FIG. 12 and the finding comment for Mark No. L1 (Mark No. 2 and Mark No. 3) illustrated in FIG. 11 is input in FIG. 13.

The finding input dialog 31 generated for the non-corresponding mark 42 includes the mark-surrounding image 25A at the supine position or the mark-surrounding image 25B at the prone position having the mark 40 affixed thereto (see FIG. 12). By using the finding input dialog 31 based on the non-corresponding mark 42, since only the other mark-surrounding image 25 is displayed, for example, the user can recognize that the feature part 13 is suspected not to be a disease or the like and can cope with this situation with an enhanced attention in observation.

The finding input dialog 31 generated for the corresponding mark 41 includes both the mark-surrounding image 25A at the supine position and the mark-surrounding image 25B at the prone position having the mark 40 affixed thereto (see FIG. 13). By using the finding input dialog 31 based on the corresponding mark 41, since both mark-surrounding images 25 are displayed, the user easily can recognize the shape or the position of the feature part 13.

The switching of the finding input dialog 31 from FIG. 12 to FIG. 13 is performed, for example, when the operation unit 120 detects that a “next mark” button is pressed. The switching of the finding input dialog 31 from FIG. 13 to FIG. 12 is performed, for example, when the operation unit 120 detects that a “previous mark” button is pressed.

The operation unit 120 receives an input of a finding comment based on the finding of the mark-surrounding image 25 including the feature part 13 having the corresponding mark 41 affixed thereto through the finding input dialog 31 (S108). The setting unit 142 sets the received finding comment and stores the setting information in the storage unit 160. The finding comment is, for example, character information but may be information other than character information.

The operation unit 120 receives an input of a finding comment based on the finding of the mark-surrounding image 25 including the feature part 13 having the non-corresponding mark 42 affixed thereto through the finding input dialog 31 (S109). The setting unit 142 sets the received finding comment and stores the setting information in the storage unit 160.

The image deriving unit 141 derives an overview image 21 at the supine position based on the volume data at the supine position and derives an overview image 21 at the prone position based on the volume data at the prone position (S110). The overview image 21 at the supine position is also referred to as an overview image 21A, and the overview image 21 at the prone position is also referred to as an overview image 21B.

In this case, the image deriving unit 141 derives the position of the mark 40 in the overview image 21A at the supine position based on the position of the mark 40 affixed to the supine image. Similarly, the image deriving unit 141 derives the position of the mark 40 in the overview image 21B at the prone position based on the position of the mark 40 affixed to the prone image.

The position of the mark 40 in various types of images (for example, the overview image 21, the virtual endoscopic image 23, the cylindrical projection image 24, and the MPR image 22) is derived, for example, based on the distance from the anus or is derived based on the distance from the central line 11 of the large intestine 10 derived from the volume data.

Plural images of the marks 40 (mark images) are stored in the storage unit 160, and the image deriving unit 141 may select different mark images as the corresponding mark 41 and the non-corresponding mark 42.

Therefore, the image deriving unit 141 derives the overview image 21A at the supine position and the overview image 21B at the prone position having the mark 40 of the selected mark image affixed thereto at the derived position. The display unit 130 displays the derived overview image 21A at the supine position and the derived overview image 21B at the prone position (S111). The mark 40 (such as the corresponding mark 41 and the non-corresponding mark 42) is affixed to the displayed overview image 21 when the mark 40 is set.

FIG. 14 is a schematic diagram illustrating an example of a finding input dialog 33 including the overview images 21 having the corresponding mark 41 and the non-corresponding mark 42 affixed thereto. The finding input dialog 33 is generated from each CT image at the supine position and the prone position and includes the overview image 21A at the supine position, the overview image 21B at the prone image, and finding comment input boxes 34 for both overview images 21A and 21B. The finding input dialog 33 is displayed subsequent to the finding input dialog 31 on the display unit 130.

Since the overview images 21 having the marks 40 (such as the corresponding mark 41 and the non corresponding mark 42) affixed thereto are displayed, the user can easily visually recognize the position of the feature part 13 suspected to be a disease in the entire large intestine 10. The user can clearly distinguish whether the correspondence relationship is set and can recognize and observe the correspondence relationship of the feature parts 13 in plural images, by checking the mark images. Particularly, when the corresponding mark 41 and the non-corresponding mark 42 are positioned close to each other, the user's attention is called to whether the set correspondence relationship is appropriate.

The operation unit 120 receives an input of a finding comment based on the finding of the overview image 12 having the corresponding mark 41 or the non-corresponding mark 42 affixed thereto through the finding input dialog 33 (S112). The setting unit 142 sets the received finding comment and stores the setting information in the storage unit 160.

When the input of the finding comment for each mark is performed and the input of the finding comment for all the marks 40 is completed, the finding input dialog 33 is derived by the dialog deriving unit 145.

The finding report generating unit 146 generates a finding report 50 based on the overview image 21A at the supine position, the overview image 21B at the prone position, and the mark-surrounding image 25 stored in the storage unit 160 and the finding comments input to the finding comment input boxes 32 and 34 (S113). When the operation unit 120 detects that a “creation of report” button on the finding input dialog 33 is pressed, the finding report generating unit 146 generates a finding report 50.

FIGS. 15 and 16 are schematic diagrams illustrating an example of the finding report 50. The finding report 50 includes, for example, bibliographic information 51, the overview image 21A at the supine position, the overview image 21B at the prone position, and finding comments 52 based on the findings of the overview images 21. For example, the corresponding mark 41 or the non-corresponding mark 42 is affixed to the overview images 21A and 21B at the supine position and the prone position.

The finding report 50 includes the mark-surrounding image 25A at the supine position, the mark-surrounding image 25B at the prone position, and the finding comments based on the mark-surrounding images 25 for each set mark 40. Here, depending on whether the correspondence relationship is set, at least one of the mark-surrounding image 25A at the supine position and the mark-surrounding image 25B at the prone position is included in the finding report 50. When plural marks 40 are affixed, plural mark-surrounding images 25 and plural finding comments 53 are present.

The layout of the finding report 50 is not limited to the layouts illustrated in FIGS. 15 and 16 but may be another layout.

FIG. 17 is a schematic diagram illustrating another layout example of the finding report 50. In FIG. 17, marks 40 (Mark 1, Mark 2, and Mark 3) are affixed. In the drawing, Mark 1 is affixed to the supine image but is not affixed to the prone image. Mark 2 is not affixed to the supine image but is affixed to the prone image. Mark 3 is affixed to both the supine image and the prone image.

Comparing FIGS. 15 and 16 with FIG. 17, in FIGS. 15 and 16, the mark-surrounding image 25A at the supine position and the mark-surrounding image 25B at the prone position to which the corresponding mark 41 is affixed are arranged vertically, and the mark-surrounding images 25A at the supine position or the mark-surrounding images 25B at the prone position are arranged horizontally. On the other hand, in FIG. 17, the mark-surrounding image 25A at the supine position and the mark-surrounding image 25B at the prone position to which the corresponding mark 41 is affixed are arranged horizontally, and the mark-surrounding images 25A at the supine position or the mark-surrounding images 25B at the prone position are arranged vertically.

In this case, the layout of FIG. 17 can be considered to be a more rational layout than the layout of FIGS. 15 and 16, in that origins of the images can be intuitively understood, because the images at the supine position are arranged on the left side and the images at the prone position are arranged on the right side.

The finding report output unit 147 transmits the derived finding report 50 to the finding server 300 or the printing machine 400. The finding report output unit 147 may output the finding report 50 to the display unit 130 or an external storage medium (not illustrated). The finding report 50 is checked, for example, in the form of electronic data or in the form of a print on a paper medium by the user.

According to the operation example of the medical image processing device 100, the user can grasp the correspondence relationship between the feature parts 13 in the images at plural body positions at a view by checking the finding report 50. Accordingly, in comparison with a case in which marks 40 are simply affixed to feature parts 13, it is possible to easily grasp the correspondence relationship between the feature parts 13. It is also possible to improve diagnosis accuracy of a doctor.

By setting the correspondence relationship, it is possible to suppress a difference in diagnosis between the doctors and to communize findings based on a medical image.

By displaying the corresponding mark 41 and the non-corresponding mark 42 in different display expressions, the user can more easily grasp the corresponding feature pans 13 in plural images.

In FIG. 6, the processes of S101 to S113 are performed in this order, but this order may be changed. For example, after the marks 40 are set in S104, the mark-surrounding images 25 may be derived in S106 and the correspondence relationship of the mark-surrounding images 25 may be set in S104. For example, when the user checks the overview image 21 but sees an error in affixing the marks 40 or setting the correspondence relationship, the processes may be repeatedly performed from S104.

When the correspondence relationship is set in S105 and the position at the supine position and the position at the prone position of the mark 40 for which the correspondence relationship is set are separated by a predetermined distance or more, the distance deriving unit 151 may output warning information including the meaning that the position of the mark 40 at the supine position and the position of the mark 40 at the prone position are separated.

The output form of warning information includes, for example, display on the display unit 130, output by voice from a voice output unit (not illustrated), and lighting or flashing of a light emitting diode (LED).

Accordingly, the medical image processing device 100 can inform the user of the possibility that the marks between which the correspondence relationship is set may not correspond to each other.

The registration process in the medical image processing device 100 will be described below.

The registration processing unit 148 may perform a registration process, for example, before the supine image and the prone image are derived in S103 of FIG. 6. The registration processing unit 148 registrates the volume data or the image at the supine position with the volume data or the image at the prone position by registration.

The registration technique in the field of colonography at the time of filing the present invention is incomplete, and the registration result can merely be used as reference information in this embodiment. This is because the large intestine 10 is greatly deformed with a change in body position, particularly, a part of the large intestine 10 is closed in images or the like, so that the passage in appearance is frequently shorted or a part of the large intestine 10 is frequently missed from the large intestine region. Accordingly, an attempt to automatically register the feature parts 13 is considered not to be practical at the time of filing the present invention. However, since the registration of peripheral tissues such as an anus or lungs is possible, the rough positional relationship of the large intestine 10 can be made to correspond thereto.

The registration includes, for example, rigid image registration (also referred to as “rigid model”) in which deformation of an image is not permitted and deformable image registration (also referred to as “deformable model”) in which deformation of an image permitted. Here, since the large intestine 10 is deformed time to time, the deformable model is preferably used in this embodiment.

The registration processing unit 148 may perform the registration, for example, using the algorithm of the deformable model (for example, see U.S. Pat. No. 8,311,300 B). As described above, it is expected to put registration employing a so-called large deformable model, instead of the simple deformable model, into practical use.

The registration processing unit 148 may registrate the cylindrical projection images at the supine position and the prone position using the technique described in the following document: Holger R. Roth, Jamie R. McClelland, Darren J. Boone, Marc Modat, M. Jorge Cardoso, Thomase. Hampshire, Mingxing Hu, Shonit Punwani, Sebastien Ourselin, Greg G. Slabaugh, Steve Halligan and David J. Hawkes, “Registration of the endoluminal surfaces of the colon derived from prone and supine CT colonography”, The International Journal of Medical Physics Research and Practice, Volume 38, Number 6, February, 2011.

The registration processing unit 148 may perform the registration using the ratio of the distances from an ileocecal valve to an anus in the volume data at the supine position and the volume data at the prone position, for example, after the passage of the large intestine 10 is derived. For example, when the tissues of the large intestine 10 are greatly deformed, it may be difficult to achieve the registration in spite of using the algorithm of the deformable model. Particularly, when the large intestine 10 is broken in images, the large intestine 10 may be determined to be cut into pieces. Even in this case, the registration processing unit 148 can perform registration using the above-mentioned ratio. This is a simple technique but is advantageous for setting the positional relationship in the passage of the large intestine 10.

By performing the registration, it is possible to match the directions of the supine image and the prone image and to enable a user to easily recognize the positional relationship between plural images. By setting the positional relationship in the passage of the large intestine 10, it is easy to estimate presence of a correspondence relationship between feature parts 13. Accordingly, a user can easily affix marks 40 to the feature parts 13 and can easily perform an operation of setting correspondence regions while watching plural images. As a result, the medical image processing device 100 can improve correspondence relationship setting accuracy and thus to improve image interpretation accuracy using a medical image.

The cleansing process of the medical image processing device 100 will be described below.

The cleansing unit 149 virtually removes the region of the residue 15 in the large intestine 10 extracted by the region extracting unit 143 using a known method (for example, see US 2007/0183644 A). The cleansing unit 149 may perform the cleansing process before the supine image and the prone image are derived in S103 of FIG. 6.

FIG. 18 is a schematic diagram illustrating an example of a virtual endoscopic image 23 subjected to the cleansing process. FIG. 19 is a schematic diagram illustrating an example of a virtual endoscopic image 23 not subjected to the cleansing process. For example, as illustrated in FIG. 19, the cleansing unit 149 virtually removes the region of the residue 15 (for example, a region in which water is estimated to be present) included in a part of a feature part 13 illustrated in FIG. 18.

When the residue 15 is removed, the cleansing unit 149 may store the region of the residue 15 in the storage unit 160. When the mark 40 is affixed in S104 of FIG. 6 and the position to which the mark 40 is affixed is included in the removed region of the residue 15 with reference to the storage unit 160, the cleansing unit 149 may output warning information including the meaning that the position to which the mark 40 is affixed is included in the removed region of the residue 15. The output form of warning information includes, for example, display on the display unit 130, output by voice from a voice output unit (not illustrated), and turning-on or turning off of an LED.

The cleansing unit 149 can extract the residue region 15 and remove the residue 15 virtually through the cleansing process, but may not always reconstruct the shape underneath the removed residue 15 in the image. Accordingly, when a feature part 13 is included in the cleansed region, the reliability with which the user determines the feature part 13 is lower than outside of the residue 15 region. In the region of the residue 15, the feature part 13 may be missed.

Therefore, since the medical image processing device 100 can affix a mark 40 to a feature part 13 outside the residue 15 and can set the correspondence relationship in a visible situation in which the residue 15 is absent, it is possible to improve extraction accuracy of the feature part 13. The medical image processing device 100 can inform the user that determination reliability of a feature part 13 including a residue 15 is low and can promote the user to carefully observe the feature part 13 by outputting the warning information for the feature part 13. When a mark 40 is affixed to a feature part 13 not including a residue 15 in a supine image and a corresponding part in a prone image is included in the region of the residue 15, the medical image processing device 100 can promote the user to carefully observe the feature part by informing the user of the possibility that the feature part 13 is included in the region of the residue 15. Whether the corresponding part is included in the region of the residue 15 may be determined by comparing the distances in the passage of the large intestine 10, or may be determined by performing precise calculation using the registration result.

The projection directions of the mark-surrounding image 25 at a supine position and a prone position will be described below.

The image deriving unit 141 determines the projection direction of the mark-surrounding image 25, that is, determines in which direction the user should observe the large intestine, based on coordinate information included in the volume data and derives the mark-surrounding image 25. Accordingly, the user can observe an arbitrary region in a desired direction or at a desired position by observing the mark-surrounding image 25.

The image deriving unit 141 may derive the mark-surrounding image 25A at the supine position and the mark-surrounding image 25B at the prone position such that the projection directions thereof match each other to have a predetermined relationship. The predetermined relationship is, for example, a relationship in which the projection directions of the mark-surrounding image 25A at the supine position and the mark-surrounding image 25B at the prone position are the same when viewed from the central line 11 of the large intestine 10. The same projection direction is a direction with respect to a human body. The projection direction at the prone position only has to be rotated by 180° relative to the projection direction at the supine position, or the projection directions may be matched by simply performing the registration. It can be considered that the registration is performed locally. Accordingly, it is possible to easily confirm that the same feature part 13 is observed at the supine position and the prone position and it is possible to easily determine whether the correspondence relationship of the marks 40 is appropriate. In addition, the medical image processing device 100 may acquire information of the same direction by matching the coordinate systems or may acquire the information of the same direction by performing more precise calculation using the registration result.

The projection direction of the mark-surrounding image 25 may be changed by an input operation to the operation unit 120. Accordingly, by changing the projection direction of the mark-surrounding image 25 displayed on the display unit 130, the user can observe the feature part 13 in an arbitrary direction. When the operation unit 120 receives an operation of changing the projection direction of the mark-surrounding image 25, the image deriving unit 141 re-derives the image deriving unit 141 based on the changing operation and displays the result on the display unit 130. When the mark-surrounding images 25 of a single feature part 13 formed in plural directions are derived, this is helpful to the diagnosis. At this time, more preferably, the medical image processing device 100 can derive pairs of mark-surrounding images 25 formed in plural directions at the supine position and the prone position.

By inputting the operation of changing the projection direction of the mark-surrounding image 25 to the operation unit 120, when a difference greater than a predetermined reference occurs between the projection direction of the mark-surrounding image 25A at the supine position and the projection direction of the mark-surrounding image 25B at the prone position which are set to a correspondence relationship, the passage deriving unit 144 may output warning information including the meaning that the difference occurs. The predetermined reference is, for example, a predetermined angle. Accordingly, it is possible to inform the user of the possibility that the derived mark-surrounding images 25 at the supine position and the prone position indicate different feature parts 13 and the correspondence relationship is not appropriate. It can be considered that the reference angle is taken to be greater in the vicinity of the curved passage of the large intestine 10. This is because the feature part 13 may move greatly at the curved position. The output form of warning information includes, for example, display on the display unit 130, output by voice from a voice output unit (not illustrated), and turning-on or turning off of an LED.

By outputting the warning information, the medical image processing device 100 can suggest whether the correspondence relationship between the mark-surrounding image 25A at the supine position and the mark-surrounding image 25B at the prone position is appropriate while the user performs the operation of designating the passage direction of the large intestine 10. Accordingly, the user can easily grasp the positional relationship between both images and can easily perform the correspondence relationship setting operation, and it is thus possible to improve correspondence relationship setting accuracy.

In this way, according to the medical image processing device 100, since the user can set the correspondence relationship between plural feature parts 13 as an observation target from the images captured at plural body positions, for example, it is possible to share and input a finding comment and it is thus possible to improve convenience to the user. The user can easily recognize relevant feature parts 13 in plural images by confirming the marks 40 having a correspondence relationship set thereto. The user can verify feature parts 13 in plural images in various directions and can easily find a disease in the feature parts 13. Accordingly, the medical image processing device 100 can improve image interpretation accuracy using a medical image.

The present invention is not limited to the configuration of the above-mentioned embodiment, but can employ any configuration as long as the configuration can achieve the functions described in the appended claims or the functions of the configuration of this embodiment.

In the above-mentioned embodiment, an image is captured and the volume data including internal information of a living body is generated using the CT equipment 200, but an image may be captured and volume data may be generated using another equipment (for example, MRI equipment).

In the above-mentioned embodiment, an image is captured at two body positions using the CT equipment 200, but an image may be captured at three or more body positions.

In the above-mentioned embodiment, the correspondence relationship is set for the marks 40 of the same feature part 13 in the supine image and the prone image. The same feature part 13 includes a variation due to deformation with a change in body position. The same feature part varies due to respiration except for the change in body position.

In the above-mentioned embodiment, the correspondence relationship setting operation is received by the operation unit 120, but the setting of a correspondence relationship may be performed through an operation of the control unit 140 using a known method. That is, the setting of a correspondence relationship may be automatically performed, not manually. After the correspondence relationship is automatically set, the user may check and correct the setting of the correspondence relationship.

In the above-mentioned embodiment, the mark images affixed to the overview images 21A and 21B at the supine position and the prone position may be displayed on the display unit 130 so as to indicate the passage direction (for example, the upstream side (small intestine side) or the downstream side (anus side) of the large intestine 10) and may appear in the finding report 50. FIG. 20 is a schematic diagram illustrating an example of a mark image of a mark 40 indicating the upstream side of the large intestine 10. In this case, the image deriving unit 141 generates a mark image based on the passage of the large intestine 10. Accordingly, the user can easily confirm that the same feature part 13 at the supine position and the prone position is observed and can easily determine whether the correspondence relationship between the marks 40 is appropriate.

In the above-mentioned embodiment, the setting unit 142 may measure the sizes of the feature parts 13, may compare the size of the feature part 13 at the supine position and the size of the feature part 13 at the prone position which are set to the correspondence relationship with each other, and may output warning information including the meaning that a difference occurs when the difference (or ratio) equal to or greater than a predetermined reference occurs therebetween.

Accordingly, the medical image processing device 100 can point out the possibility that the feature part 13 observed at the supine position and the feature part 13 observed at the prone position indicate different feature parts 13 and the correspondence relationship therebetween is not appropriate. Here, since a slight error is predicted depending on imaging conditions or measurement accuracy, the sizes of both feature parts 13 does not need to be equal to each other. As the size of a feature part 13, the size, the region, the volume, the minor diameter, the major diameter, and the like of the feature part 13 can be considered. A combination thereof may be set as a condition. The output form of warning information includes, for example, display on the display unit 130, output by voice from a voice output unit (not illustrated), and turning-on or turning off of an LED.

### Exemplary Embodiments of Invention

According to one aspect of the present invention, a medical image processing device of the present invention includes an acquisition unit, an image deriving unit, a setting unit, an input unit and a report generating unit. The acquisition unit acquires first volume data including a digestive organ which is imaged in a first body position and acquires second volume data including the digestive organ which is imaged in a second body position. The image deriving unit derives a first image including the digestive organ based on the first volume data and derives a second image including the digestive organ based on the second volume data. The setting unit sets a first mark for a first feature part included in the first volume data, sets a second mark for a second feature part included in the second volume data, sets a third mark for a third feature part included in the second volume data, and sets a correspondence relationship indicating that the first mark and the second mark correspond to each other. The input unit inputs first finding information based on a common finding about the first feature part having the first mark and the second feature part having the second mark. The first and second marks are set to the correspondence relationship. The input unit inputs second finding information based on an individual finding about the third feature part having the third mark. The report generating unit generates a finding report which includes the first image, the second image, the first and second marks set to the correspondence relationship, the first finding information, the third mark, and the second finding information, in which the first mark and the second mark are displayed in a same expression, and in which the first mark and the third mark are displayed in a different expression.

According to this configuration, in the medical image processing device, since a user can set a correspondence relationship between plural feature pans as an observation target from the images captured at plural body positions, for example, it is possible to share the input of finding comment and it is thus possible to improve convenience to the user. The user can easily recognize relevant feature parts in plural images by confirming the marks having a correspondence relationship set thereto. The user can verify feature parts in plural images in various directions and can easily find a disease in the feature parts. The user can clearly discriminate a mark having a correspondence relationship set thereto and a mark not having a correspondence relationship set thereto by checking the find report and suppress misunderstanding of the correspondence relationship. The user can determine whether the correspondence relationship is appropriate at a look. For example, when a first mark and a second mark having a correspondence relationship set thereto are separated a long distance (equal to or greater than a predetermined distance), the user can recognize the possibility that the correspondence relationship will be set erroneously. When a second mark and a third mark are present within a short distance (within a predetermined distance), for example, the user can recognize the possibility that the correspondence relationship is set erroneously or a missing is present. Accordingly, the medical image processing device can improve image interpretation accuracy using a medical image.

In the medical image processing device according to the present invention, the first body position is a supine position and the second body position is a prone position.

According to this configuration, an image of a patient can be easily captured at plural body positions and vokune data at plural body positions can be acquired.

The medical image processing device according to the present invention may further include a mark-surrounding image generating unit. The mark-surrounding image generating unit generates a first mark-surrounding image including the first feature part and the first mark based on the first volume data, generates a second mark-surrounding image including the second feature part and the second mark based on the second volume data, and generates a third mark-surrounding image including the third feature part and the third mark based on the second volume data. The report generating unit generates the finding report including the first mark-surround image, the second mark-surrounding image, and the third mark-surrounding image.

According to this configuration, for example, it is possible to easily visually recognize a set mark or a set correspondence relationship and to generate a mark-surrounding image.

In the medical image processing device according to the present invention, the mark-surrounding image generating unit generates the first mark-surrounding image and the second mark-surrounding image in a same projection direction.

According to this configuration, the first mark-surrounding image and the second mark-surrounding image can be displayed in the finding report in correlation which each other, and the user can easily grasp the relationship between the position in the first mark-surrounding image and the position in the second mark-surrounding image. Accordingly, the medical image processing device 100 can improve image interpretation accuracy by confirming the correspondence relationship in the finding report.

The medical image processing device according to the present invention may further include an output unit. The output unit outputs warning information if a first projection direction and a second projection direction satisfy a predetermined condition. The first mark-surrounding image is generated in the first projection direction and the second mark-surrounding image is generated in the second projection direction.

According to this configuration, the user can perform an operation of reducing a difference in projection direction between the first mark-surrounding image and the second mark-surrounding image, for example, in the course of generating the finding report by confirming the output warning information. Accordingly, the medical image processing device can suppress the difficulty in grasping the relationship between the position in the first mark-surrounding image and the position in the second mark-surrounding image because the difference in projection direction between the first mark-surrounding image and the second mark-surrounding image increases in the course of generating the finding report. As a result, the medical image processing device 100 can improve correspondence relationship setting accuracy and can improve image interpretation accuracy using a medical image.

The medical image processing device according to the present invention may further include a reference line deriving unit. The reference line deriving unit derives a first reference line along a passage of the digestive organ based on the first volume data and derives a second reference line along the passage of the digestive organ based on the second volume data. The report generating unit generates the finding report including the first reference line with the first image and including the second reference line with the second image.

According to this configuration, since the user can easily recognize the passage of the digestive organ, the user can easily recognize the position of a mark in the digestive organ. Accordingly, the medical image processing device can improve image interpretation accuracy using the finding report.

The medical image processing device according to the present invention may further include a passage deriving unit. The passage deriving unit derives a first passage of the digestive organ based on the first volume data and derives a second passage of the digestive organ based on the second volume data. The report generating unit visualizes the direction of the first passage in the first feature part using the first mark, and visualizes the direction of the second passage in the second feature part using the second mark.

According to this configuration, the user can recognize the passage of the digestive organ in a feature part by confirming the mark in the finding report and can interpret the image in consideration of the passage. Accordingly, the medical image processing device can improve image interpretation accuracy using the finding report.

The medical image processing device according to the present invention may further include: a passage deriving unit and a distance deriving unit. The passage deriving unit derives a first passage of the digestive organ based on the first volume data and derives a second passage of the digestive organ based on the second volume data. The distance deriving unit derives a first distance which is a distance between a first reference position and the position of the first feature part in the first volume data based on the first passage and derives a second distance which is a distance between a second reference position and the position of the second feature part in the second volume data based on the second passage. The report generating unit generates the finding report including the first distance and the second distance.

According to this configuration, the user can easily recognize the position of the mark with respect to a predetermined reference position by confirming information of the first distanced and the second distance in the finding report. Accordingly, the medical image processing device can improve image interpretation accuracy using the finding report.

The medical image processing device according to the present invention may further include an output unit. The output unit outputs warning information when a difference between the first distance and the second distance is equal to or greater than a predetermined difference.

According to this configuration, the medical image processing device can inform the user of a high possibility that the first mark and the second mark will not correspond to each other when the first mark and the second mark are separated a relatively large distance. Accordingly, since the medical image processing device can suppress the setting of a correspondence relationship between the marks not corresponding to each other in the course of generating the finding report, it is possible to improve the correspondence relationship setting accuracy. Accordingly, the medical image processing device can improve image interpretation accuracy using the finding report.

The medical image processing device according to the present invention may further include: an output unit; and an extraction unit. The extraction unit extracts residue information included in the second volume data. The output unit outputs warning information when a position in the second volume data corresponding to a position of the first feature part is included in a region of the residue information.

According to this configuration, when a region of a residue is virtually deleted and the deletion accuracy is low, the user can recognize that the reliability of the mark affixed to the feature part is low by checking the warning information. Accordingly, since the medical image processing device can suppress the setting of a correspondence relationship using marks with low reliability in the course of generating the finding report, it is possible to improve the correspondence relationship setting accuracy. Accordingly, the medical image processing device can improve image interpretation accuracy using the finding report.

In the medical image processing device according to the present invention, the first image and the second image may include a virtual endoscopic image, a cylindrical projection image, or an overview image which display the digestive organ.

According to this configuration, when the first image and the second image are displayed on the display unit, the medical image processing device can display an image in various display expressions. Accordingly, the medical image processing device can select the display expression of the first image and the second image, for example, in consideration of the setting of marks or the setting of a correspondence relationship.

The medical image processing device according to the present invention may further include an output unit. The output unit outputs warning information when a first size of the first feature part and a second size of the second feature part satisfy a predetermined condition.

According to this configuration, the medical image processing device can point out the possibility that the feature part observed at the first body position and the feature part observed at the second body position indicate different feature parts and the correspondence relationship therebetween is not appropriate.

A medical image processing method in a medical image processing device according to the present invention including: acquiring first volume data including a digestive organ which is imaged in a first body position; acquiring second volume data including the digestive organ which is imaged in a second body position; deriving a first image including the digestive organ based on the first volume data; deriving a second image including the digestive organ based on the second volume data; setting a first mark for a first feature part included in the first volume data; setting a second mark for a second feature part included in the second volume data; setting a third mark for a third feature part included in the second volume data; setting a correspondence relationship indicating that the first mark and the second mark correspond to each other; inputting first finding information based on a common finding about the first feature part having the first mark and the second feature part having the second mark, the first and second marks being set to the correspondence relationship; inputting second finding information based on an individual finding about the third feature part having the third mark; and generating a finding report which includes the first image, the second image, the first and second marks set to the correspondence relationship, the first finding information, the third mark, and the second finding information, in which the first mark and the second mark are displayed in a same expression, and in which the first mark and the third mark are displayed in a different expression.

According to this method, in the medical image processing device, since a user can set a correspondence relationship between plural feature parts as an observation target from the images captured at plural body positions, for example, it is possible to share and input a finding comment and it is thus possible to improve convenience to the user. The user can easily recognize relevant feature parts in plural images by confirming the marks having a correspondence relationship set thereto. The user can verify feature parts in plural images in various directions and can easily find a disease in the feature parts. The user can clearly discriminate a mark having a correspondence relationship set thereto and a mark not having a correspondence relationship set thereto by checking the find report and suppress misunderstanding of the correspondence relationship. The user can determine whether the correspondence relationship is appropriate at a look. For example, when a first mark and a second mark having a correspondence relationship set thereto are separated a long distance (equal to or greater than a predetermined distance), the user can recognize the possibility that the correspondence relationship will be set erroneously. When a second mark and a third mark are present within a short distance (within a predetermined distance), for example, the user can recognize the possibility that the correspondence relationship is set erroneously or a missing is present. Accordingly, the medical image processing device can improve image interpretation accuracy using a medical image.

A non-transitory computer readable medium according to the present invention stores program for causing a medical image processing device to execute operations including: acquiring first volume data including a digestive organ which is imaged in a first body position; acquiring second volume data including the digestive organ which is imaged in a second body position; deriving a first image including the digestive organ based on the first volume data; deriving a second image including the digestive organ based on the second volume data; setting a first mark for a first feature part included in the first volume data; setting a second mark for a second feature part included in the second volume data; setting a third mark for a third feature part included in the second volume data; setting a correspondence relationship indicating that the first mark and the second mark correspond to each other; inputting first finding information based on a common finding about the first feature part having the first mark and the second feature part having the second mark, the first and second marks being set to the correspondence relationship; inputting second finding information based on an individual finding about the third feature part having the third mark; and generating a finding report which includes the first image, the second image, the first and second marks set to the correspondence relationship, the first finding information, the third mark, and the second finding information, in which the first mark and the second mark are displayed in a same expression, and in which the first mark and the third mark are displayed in a different expression.

According to this program, in the computer executing the program, since a user can set a correspondence relationship between plural feature parts as an observation target from the images captured at plural body positions, for example, it is possible to share and input a finding comment and it is thus possible to improve convenience to the user. The user can easily recognize relevant feature parts in plural images by confirming the marks having a correspondence relationship set thereto. The user can verify feature pans in plural images in various directions and can easily find a disease in the feature parts. The user can clearly discriminate a mark having a correspondence relationship set thereto and a mark not having a correspondence relationship set thereto by checking the find report and suppress misunderstanding of the correspondence relationship. The user can determine whether the correspondence relationship is appropriate at a look. For example, when a first mark and a second mark having a correspondence relationship set thereto are separated a long distance (equal to or greater than a predetermined distance), the user can recognize the possibility that the correspondence relationship will be set erroneously. When a second mark and a third mark are present within a short distance (within a predetermined distance), for example, the user can recognize the possibility that the correspondence relationship is set erroneously or a missing is present. Accordingly, the computer can improve image interpretation accuracy using a medical image.

The present invention can be usefully applied to a medical image processing device, a medical image processing method, and a medical image processing program which can improve image interpretation accuracy using a medical image.

What is claimed is:

1. A medical image processing device comprising:
an acquisition unit that acquires first volume data including a digestive organ which is imaged in a first body position and that acquires second volume data including the digestive organ which is imaged in a second body position;
an image deriving unit that derives a first image including the digestive organ based on the first volume data and that derives a second image including the digestive organ based on the second volume data;
a setting unit that sets a first mark for a first feature part included in the first volume data, that sets a second mark for a second feature part included in the second volume data, that sets a third mark for a third feature part included in the second volume data, and that sets a correspondence relationship indicating that the first mark and the second mark correspond to each other;
an input unit that inputs first funding information based on a common finding about the first feature part having the first mark and the second feature part having the second mark, the first and second marks being set to the correspondence relationship, and that inputs second finding information based on an individual finding about the third feature part having the third mark; and
a report generating unit that generates a finding report which includes the first image, the second image, the first and second marks set to the correspondence relationship, the first finding information, the third mark, and the second funding information, in which the first mark and the second mark are displayed in a same expression, and in which the first mark and the third mark are displayed in a different expression.

2. The medical image processing device according to claim 1,
wherein the first body position is a supine position and the second body position is a prone position.

3. The medical image processing device according to claim 1, further comprising:
a mark-surrounding image generating unit that generates a first mark-surrounding image based on the first volume data, that generates a second mark-surrounding image based on the second volume data, and that generates a third mark-surrounding image based on the second volume data,
wherein the first mark-surrounding image includes the first feature part and the first mark;
wherein the second mark-surrounding image includes the second feature part and the second mark;
wherein the third mark-surrounding image includes the third feature part and the third mark; and
wherein the report generating unit generates the finding report including the first mark-surround image, the second mark-surrounding image, and the third mark-surrounding image.

4. The medical image processing device according to claim 3,
wherein the mark-surrounding image generating unit generates the first mark-surrounding image and the second mark-surrounding image in a same projection direction.

5. The medical image processing device according to claim 3, further comprising:
an output unit that outputs warning information if a first projection direction and a second projection direction satisfy a predetermined condition,
wherein the first mark-surrounding image is generated in the first projection direction and the second mark-surrounding image is generated in the second projection direction.

6. The medical image processing device according to claim 1, further comprising:
a reference line deriving unit that derives a first reference line along a passage of the digestive organ based on the first volume data and that derives a second reference line along the passage of the digestive organ based on the second volume data,
wherein the report generating unit generates the finding report including the first reference line with the first image and including the second reference line with the second image.

7. The medical image processing device according to claim 1, further comprising:
a passage deriving unit that derives a first passage of the digestive organ based on the first volume data and that derives a second passage of the digestive organ based on the second volume data,
wherein the report generating unit visualizes a first direction of the first passage in the first feature part using the first mark, visualizes a second direction of the second passage in the second feature part using the second mark, and generates the finding report.

8. The medical image processing device according to claim 1, further comprising:
a passage deriving unit that derives a first passage of the digestive organ based on the first volume data and that derives a second passage of the digestive organ based on the second volume data;
a distance deriving unit that derives a first distance which is a distance between a first reference position and a position of the first feature part in the first volume data based on the first passage and that derives a second distance which is a distance between a second reference position and a position of the second feature part in the second volume data based on the second passage,
wherein the report generating unit generates the finding report including the first distance and the second distance.

9. The medical image processing device according to claim 8, further comprising:
an output unit that outputs warning information when a difference between the first distance and the second distance is equal to or greater than a predetermined difference.

10. The medical image processing device according to claim 1, further comprising:
an output unit; and
an extraction unit that extracts residue information included in the second volume data,
wherein the output unit outputs warning information when a position in the second volume data corresponding to a position of the first feature part is included in a region of the residue information.

11. The medical image processing device according to claim 1,
wherein the first image and the second image include a virtual endoscopic image, a cylindrical projection image, or an overview image which displays the digestive organ.

12. The medical image processing device according to claim 1, further comprising:
an output unit that outputs warning information when a first size of the first feature part and a second size of the second feature part satisfy a predetermined condition.

13. A medical image processing method in a medical image processing device, comprising:
acquiring first volume data including a digestive organ which is imaged in a first body position;
acquiring second volume data including the digestive organ which is imaged in a second body position;
deriving a first image including the digestive organ based on the first volume data;
deriving a second image including the digestive organ based on the second volume data;
setting a first mark for a first feature part included in the first volume data;
setting a second mark for a second feature part included in the second volume data;
setting a third mark for a third feature part included in the second volume data;
setting a correspondence relationship indicating that the first mark and the second mark correspond to each other;
inputting first finding information based on a common finding about the first feature part having the first mark and the second feature part having the second mark, the first and second marks being set to the correspondence relationship;
inputting second finding information based on an individual finding about the third feature part having the third mark; and
generating a finding report which includes the first image, the second image, the first and second marks set to the correspondence relationship, the first finding information, the third mark, and the second finding information, in which the first mark and the second mark are displayed in a same expression, and in which the first mark and the third mark are displayed in a different expression.

14. A non-transitory computer readable medium which stores program for causing a medical image processing device to execute operations comprising:
acquiring first volume data including a digestive organ which is imaged in a first body position;
acquiring second volume data including the digestive organ which is imaged in a second body position;
deriving a first image including the digestive organ based on the first volume data;
deriving a second image including the digestive organ based on the second volume data;
setting a first mark for a first feature part included in the first volume data;
setting a second mark for a second feature part included in the second volume data;
setting a third mark for a third feature part included in the second volume data;
setting a correspondence relationship indicating that the first mark and the second mark correspond to each other;
inputting first finding information based on a common finding about the first feature part having the first mark and the second feature part having the second mark, the first and second marks being set to the correspondence relationship;
inputting second finding information based on an individual finding about the third feature part having the third mark; and
generating a finding report which includes the first image, the second image, the first and second marks set to the correspondence relationship, the first finding information, the third mark, and the second finding information, in which the first mark and the second mark are displayed in a same expression, and in which the first mark and the third mark are displayed in a different expression.
